(19) United States
(12) Patent Application Publication
(10) Pub. No.: US 2024/0197190 A1
(43) Pub. Date: Jun. 20, 2024

(54) DEVICE AND METHOD FOR EXTRACTING CARDIOVASCULAR HEALTH STATUS BY USING OSCILLOMETRIC SIGNAL

(71) Applicant: Korea Advanced Institute of Science and Technology, Daejeon (KR)

(72) Inventors: Yong-Hwa PARK, Daejeon (KR); Bomi LEE, Daejeon (KR); Junki HONG, Daejeon (KR); Jae Hak JEONG, Daejeon (KR)

(21) Appl. No.: 18/198,397

(22) Filed: May 17, 2023

(30) Foreign Application Priority Data
Dec. 19, 2022 (KR) 10-2022-0178074

(51) Int. Cl. A61B 5/022; A61B 5/00; A61B 5/02; A61B 5/021; A61B 5/0285; A61B 5/107

(52) U.S. Cl. CPC A61B 5/02225; A61B 5/02007; A61B 5/02108; A61B 5/0285; A61B 5/1075; A61B 5/7278

(57) ABSTRACT

The present disclosure relates to a device and method for extracting a cardiovascular health status by using an oscillometric signal. The device and method may be configured to extract at least one characteristic factor of an oscillometric signal, extract a pulse wave from the oscillometric signal, and extract at least one cardiovascular health index by analyzing the characteristic factor and the pulse wave.

### CROSS-REFERENCES TO RELATED APPLICATION

This application is based on and claims priority under 35 U.S.C. 119 to Korean Patent Application No. 10-2022-0178074, filed on Dec. 19, 2022 in the Korean intellectual property office, the disclosures of which are herein incorporated by reference in their entireties.

### TECHNICAL FIELD

The present disclosure relates to a device and method for extracting a cardiovascular health status by using an oscillometric signal

### BACKGROUND OF THE DISCLOSURE

A cardiovascular disease is one of major causes of death. A major cause of the cardiovascular disease is a change in anaplastia of a cardiovascular system. Accordingly, the periodic check of a personal cardiovascular health status and the course of a change thereof may help in preventing the cardiovascular disease. A related index that is now most commonly measured is blood pressure. Blood pressure can be easily measured even in a home by using an automatic blood pressure gauge. The automatic blood pressure gauge may estimate blood pressure based on an oscillometric signal. The oscillometric signal indicates a vibration signal in which a pulse wave within a blood vessel is transferred as the air within a pressurized cuff of a blood pressure system through a vessel wall and a soft tissue of an upper arm.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

The present disclosure is intended to be used in the prevention and early diagnosis of a cardiovascular disease by enabling a personal cardiovascular health status and the course of a change thereof to be periodically easily measured through an automatic blood pressure gauge that is widely used.

The present disclosure provides a method and device capable of checking a personal cardiovascular health status and the course of a change thereof in an oscillometric signal that is obtained in a process of measuring blood pressure by using an automatic blood pressure gauge.

The present disclosure provides a device and method for extracting a cardiovascular health status by using an oscillometric signal.

In an embodiment, a method of extracting a cardiovascular health status by using an oscillometric signal may include extracting at least one characteristic factor of an oscillometric signal, extracting a pulse wave from the oscillometric signal, and extracting at least one cardiovascular health index by analyzing the characteristic factor and the pulse wave.

In an embodiment, an electronic device for extracting a cardiovascular health status by using an oscillometric signal, the electronic device may include memory, and a processor connected to the memory and configured to execute at least one instruction stored in the memory. The processor may be configured to extract at least one characteristic factor of an oscillometric signal, extract a pulse wave from the oscillometric signal, and extract at least one cardiovascular health index by analyzing the characteristic factor and the pulse wave.

In an embodiment, in a non-transitory computer-readable recording medium in which a computer program for executing a method of extracting a cardiovascular health status by using an oscillometric signal in an electronic device has been stored, the method of extracting the cardiovascular health status may include extracting at least one characteristic factor of an oscillometric signal, extracting a pulse wave from the oscillometric signal, and extracting at least one cardiovascular health index by analyzing the characteristic factor and the pulse wave.

According to the present disclosure, the electronic device can extract a cardiovascular health status by using an oscillometric signal. That is, the electronic device can check a cardiovascular health status and the course of a change thereof by analyzing an oscillometric signal that is detected by using a common automatic blood pressure gauge. Accordingly, the electronic device may be applied to all fields in which the automatic blood pressure gauge is used. For example, when blood pressure of a patient who periodically visits a hospital is measured by using the automatic blood pressure gauge, the electronic device can determine a cardiovascular disease by extracting a cardiovascular health status in addition to blood pressure and checking the course of a change thereof. As another example, when the automatic blood pressure gauge is periodically used in a home, the electronic device can determine a cardiovascular disease by extracting a cardiovascular health status in addition to blood pressure and checking the course of a change thereof. Accordingly, a cardiovascular disease can be prevented and early diagnosed. Moreover, even people whose blood pressure is measured high due to a white coat syndrome when the blood pressure is measured in a hospital can check their cardiovascular health statuses in a comfortable environment, such as a home.

Furthermore, patients who belong to a high risk group for a cardiovascular disease can previously recognize the dangerousness of the cardiovascular disease through consistent measurement even in homes. Such an electronic device may be applied to all types of devices which detect an oscillometric signal, such as an automatic blood pressure gauge.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a diagram schematically illustrating a construction of an electronic device according to various embodiments.

FIG. 2 is a diagram for exemplarily describing an operating characteristic of the electronic device according to various embodiments.

FIG. 3 is a diagram specifically illustrating a construction of a processor illustrated in FIG. 1.

FIG. 4 is a diagram for exemplarily describing an oscillometric signal in various embodiments.

FIGS. 5, 6, 7, and 8 are diagrams for exemplarily describing the extraction of characteristic factors of an oscillometric signal in various embodiments.

FIG. 9 is a diagram for exemplarily describing the extraction of a pulse wave from an oscillometric signal in various embodiments.

FIGS. 10, 11, and 12 are diagrams for exemplarily describing the analysis of a pulse wave in various embodiments.

FIG. 13 is a diagram schematically illustrating an operating method of the electronic device according to various embodiments.

### DETAILED DESCRIPTION

While illustrative embodiments have been illustrated and described, it will be appreciated that various changes can be made therein without departing from the spirit and scope of the disclosure.

Hereinafter, various embodiments of the present disclosure are described with reference to the accompanying drawings.

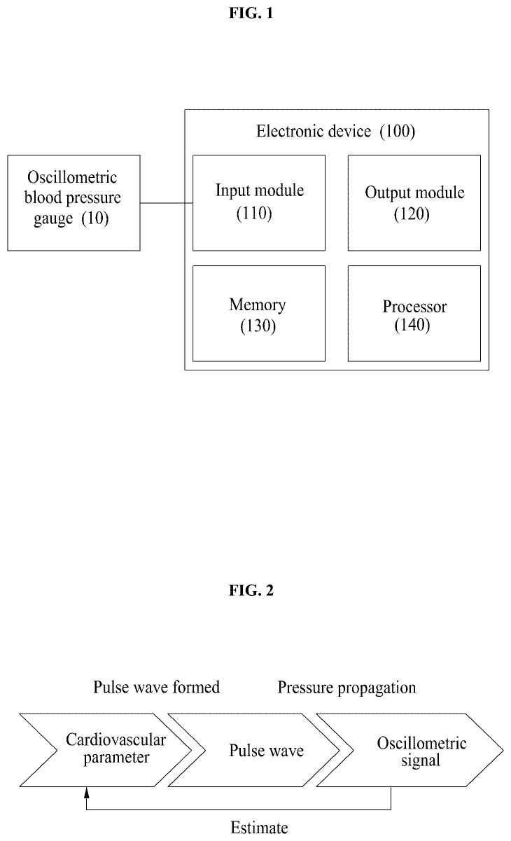

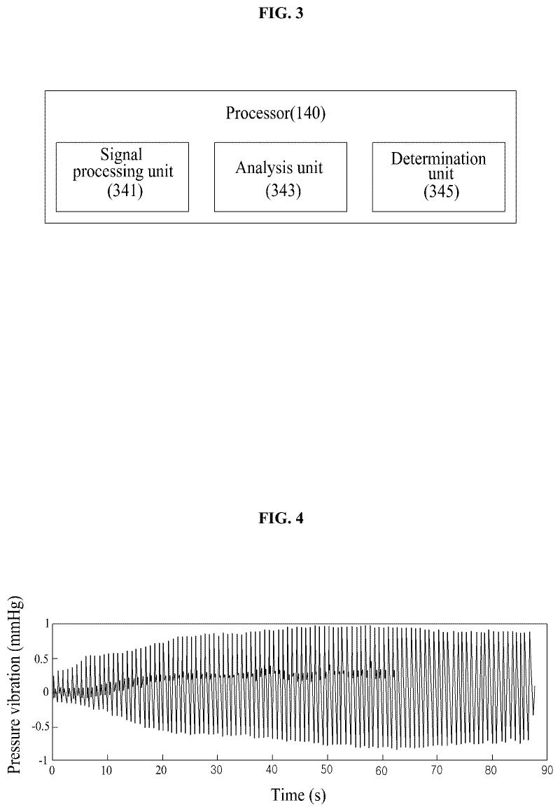

FIG. 1 is a diagram schematically illustrating a construction of an electronic device 100 according to various embodiments. FIG. 2 is a diagram for exemplarily describing an operating characteristic of the electronic device 100 according to various embodiments.

Referring to FIG. 1, the electronic device 100 may be provided to extract a cardiovascular health status by using an oscillometric signal. To this end, an oscillometric blood pressure gauge 10 may be integrated into the electronic device 100, or may be configured separately from the electronic device 100. The electronic device 100 may include at least one of an input module 110, an output module 120, memory 130, or a processor 140. In an embodiment, at least one of the components of the electronic device 100 may be omitted, and at least another component may be added to the electronic device 100. In an embodiment, at least two of the components of the electronic device 100 may be implemented as one integrated circuit.

The oscillometric blood pressure gauge 10 may be configured to estimate blood pressure of a blood vessel by using a cuff that is mounted on the upper arm or wrist of a user. Specifically, as a pulse wave of a blood vessel is transferred as the air within the cuff through a vessel wall and a soft tissue in response to cuff pressure that slowly relieves air pressure after applying the air pressure to the cuff, fine pressure vibration may be generated in the cuff. The oscillometric blood pressure gauge 10 may detect an oscillometric signal from the pressure vibration in the cuff. Furthermore, the oscillometric blood pressure gauge 10 may estimate blood pressure of the blood vessel by using the size of the pressure vibration.

The input module 110 may input a signal to be used in at least one component of the electronic device 100. The input module 110 may be configured to detect a signal that is directly input by a user or to generate a signal by detecting a surrounding change. For example, the input module 110 may include at least one of a microphone, at least one physical button, or a touch pad. The input module 110 may include a reception device configured to receive a signal from an external device. According to an embodiment, the input module 110 may be directly connected to the oscillometric blood pressure gauge 10, and may receive at least one of an oscillometric signal or measured blood pressure from the oscillometric blood pressure gauge 10. According to another embodiment, the input module 110 may receive at least one of an oscillometric signal or measured blood pressure from another electronic device. The reception device will be more specifically described later.

The output module 120 may output information to the outside of the electronic device 100. The output module 120 may be implemented as a tactile display for providing a tactile interface. To this end, the output module 120 may include at least one of a display module configured to visually output information or an audio output module configured to acoustically output information. For example, the audio output device may include at least one of a speaker or a receiver. The output module 120 may include at least one transmission device capable of wirelessly transmitting information. The transmission device will be more specifically described later.

According to some embodiments, the reception device and the transmission device may be implemented as a communication module. The communication module may perform communication with an external device in the electronic device 100. The communication module may establish a communication channel between the electronic device 100 and the external device, and may perform communication with the external device through the communication channel. The communication module may include at least one of a wired communication module or a wireless communication module. The wired communication module is connected to the external device in a wired way, and may communicate with the external device in a wired way. The wireless communication module may include at least one of a short-distance communication module or a long-distance communication module. The short-distance communication module may communicate with the external device using the short-distance communication method. For example, the short-distance communication method may include at least one of Bluetooth, Wi-Fi direct, or infrared data association (IrDA). The long-distance communication module may communicate with the external device using the long-distance communication method. In this case, the long-distance communication module may communicate with the external device over a network. For example, the network may include at least one of a cellular network, the Internet, or a computer network, such as a local area network (LAN) or a wide area network (WAN).

The memory 130 may store various data that are used by at least one component of the electronic device 100. For example, the memory 130 may include at least one of a volatile memory or a nonvolatile memory. The data may include at least one program and input data or output data related thereto. The program may be stored in the memory 130 as software including at least one instruction, and may include at least one of an operating system, middleware, or an application.

The processor 140 may control at least one component of the electronic device 100 by executing a program of the memory 130. Accordingly, the processor 140 may perform data processing or an operation. In this case, the processor 140 may execute an instruction stored in the memory 130.

As described above, the oscillometric signal may be detected from pressure vibration that is generated in a cuff as a pulse wave of a blood vessel is transferred to the cuff through a vessel wall and a soft tissue in response to cuff pressure. As illustrated in FIG. 2, a pulse wave that is the base of an oscillometric signal is formed as the sum of an advancing wave that is ejected from the heart and a reflection wave that is reflected by a branch point. The size and a shape of such a pulse wave may be changed due to cardiovascular parameters, for example, the elasticity of a blood vessel, the internal diameter of a blood vessel, and peripheral resistance. Accordingly, when a cardiovascular parameter is changed over a flow of time due to aging, a pulse wave may be changed, and an oscillometric signal may also be changed. Accordingly, the processor 140 may check a cardiovascular health status by inversely estimating a cardiovascular parameter from an oscillometric signal.

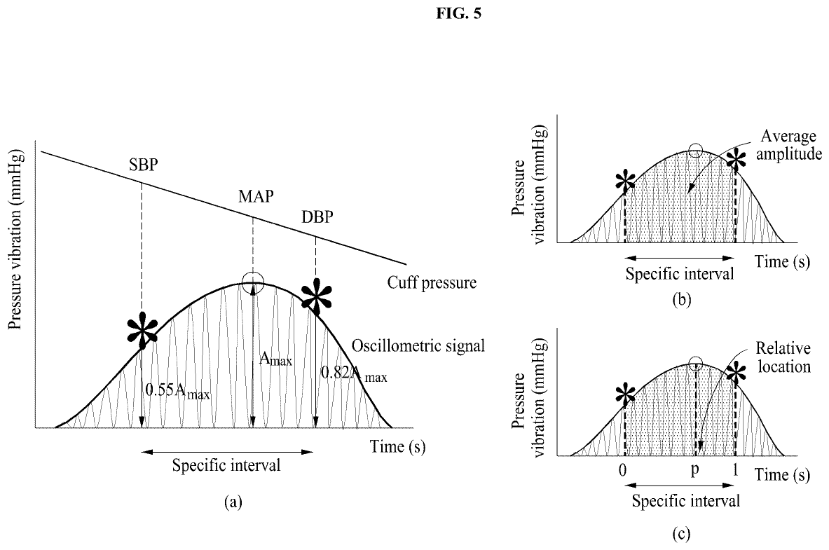

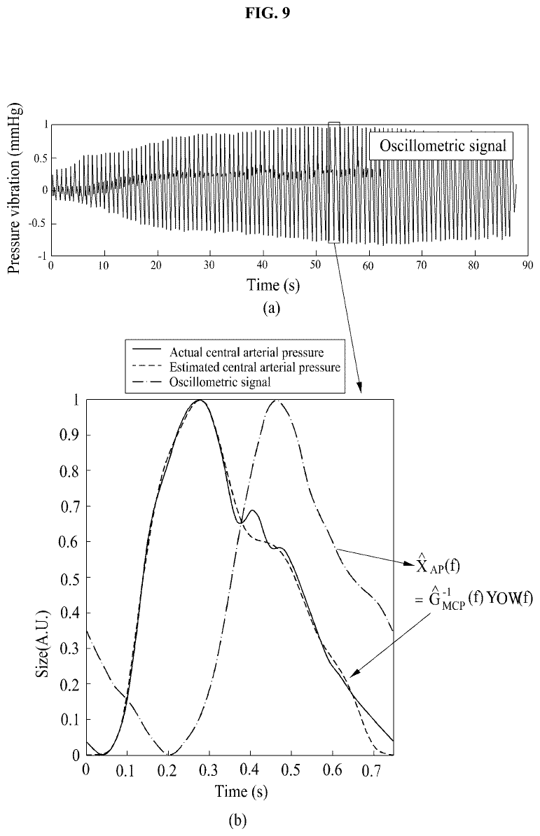

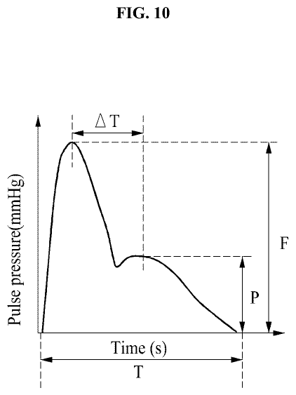

FIG. 3 is a diagram specifically illustrating a construction of the processor 140 illustrated in FIG. 1. FIG. 4 is a diagram for exemplarily describing an oscillometric signal in various embodiments. FIGS. 5, 6, 7, and 8 are diagrams for exemplarily describing the extraction of characteristic factors of an oscillometric signal in various embodiments. FIG. 9 is a diagram for exemplarily describing the extraction of a pulse wave from an oscillometric signal in various embodiments. FIGS. 10, 11, and 12 are diagrams for exemplarily describing the analysis of a pulse wave in various embodiments.

Referring to FIG. 3, the processor 140 may include a signal processing unit 341, an analysis unit 343, and a determination unit 345.

The signal processing unit 341 may detect an oscillometric signal. In this case, when the oscillometric blood pressure gauge 10 detects the oscillometric signal that is generated in response to cuff pressure, the signal processing unit 341 may detect the oscillometric signal as illustrated in FIG. 4.

Furthermore, the signal processing unit 341 may extract at least one characteristic factor of the oscillometric signal. The characteristic factor may include at least one of the amplitude, type, or frequency component of the oscillometric signal.

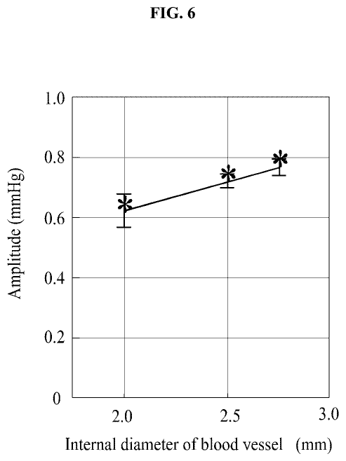

According to an embodiment, the signal processing unit 341 may extract the amplitude of the oscillometric signal as a characteristic factor. For example, as illustrated in FIG. 5(a), the signal processing unit 341 may set a specific section for the oscillometric signal. In this case, the specific section may indicate a section between systolic blood pressure (SBP) and diastolic blood pressure (DBP) according to the cuff pressure. Furthermore, as illustrated in FIG. 5(b), the signal processing unit 341 may extract average amplitude of the oscillometric signal within the specific section as a characteristic factor. In this case, as illustrated in FIG. 6, the amplitude of the oscillometric signal may have a correlation with an internal diameter of a blood vessel. That is, as the internal diameter of the blood vessel is increased, the amplitude of the oscillometric signal may be further increased.

According to another embodiment, the signal processing unit 341 may extract the amplitude and type of the oscillometric signal as characteristic factors. For example, as illustrated in FIG. 5(a), the signal processing unit 341 may set the specific section for the oscillometric signal, and may extract a maximum amplitude point (MAP) of the oscillometric signal within the specific section. Furthermore, the signal processing unit 341 may extract at least one of average amplitude of the oscillometric signal or maximum amplitude of the oscillometric signal at the maximum amplitude point of the oscillometric signal within the specific section as characteristic factors as illustrated in FIG. 5(b), and may extract a relative location for the maximum amplitude point of the oscillometric signal within the specific section as a characteristic factor as illustrated in FIG. 5(c).

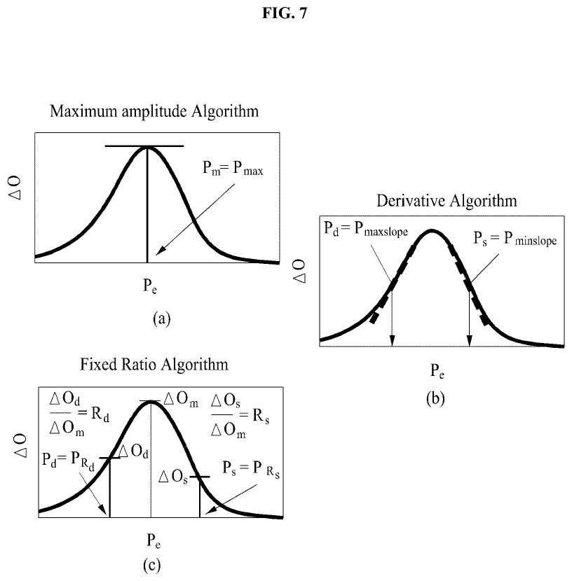

According to still another embodiment, the signal processing unit 341 may extract the type of oscillometric signal as a characteristic factor by using various methods. For example, as illustrated in FIG. 7(a), the signal processing unit 341 may extract maximum amplitude point (Pm) of the oscillometric signal, and may extract the location of the maximum amplitude point of the oscillometric signal as a characteristic factor. As another example, as illustrated in FIG. 7(b), the signal processing unit 341 may extract a maximum value (Pd) and minimum value (Ps) of primary and secondary differential values from the oscillometric signal, and may extract the maximum value, the minimum value, and locations thereof as characteristic factors. As still another example, as illustrated in FIG. 7(c), the signal processing unit 341 may extract a first point (Pd) corresponding to amplitude of the oscillometric signal having a predetermined first ratio (Rd) with respect to the amplitude of the oscillometric signal at the maximum amplitude point prior to the maximum amplitude point, and a second point (Ps) corresponding to amplitude of the oscillometric signal having a predetermined second ratio (Rs) with respect to amplitude of the oscillometric signal at the maximum amplitude point after the maximum amplitude point, and may extract locations thereof as characteristic factors.

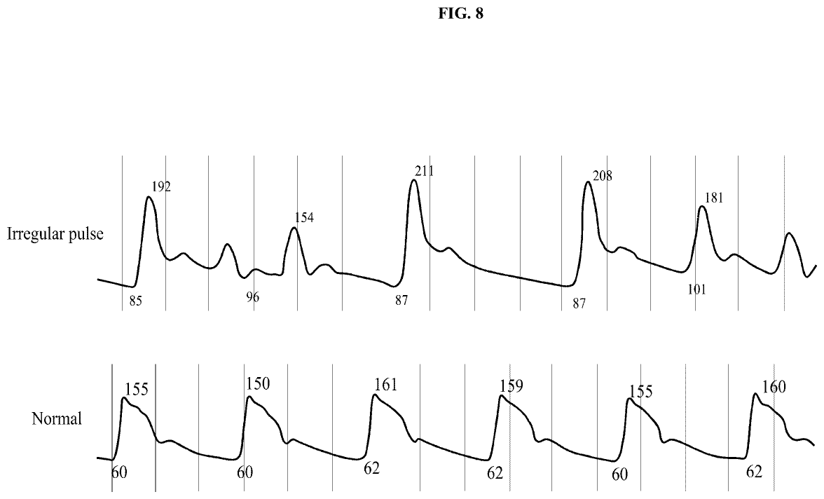

According to still another embodiment, the signal processing unit 341 may extract, as a characteristic factor, a frequency component indicating a form of a frequency spectrum of the oscillometric signal. In this case, as illustrated in FIG. 8, the frequency component may be divided into multiple periods depending on a pulse thereof. For example, when a pulse is normal, the frequency component may have uniform periodicity, and an abnormal frequency portion other than the pulse is not present in each period. When the pulse is abnormal like an irregular pulse, the frequency component may have nonuniform periodicity, and an abnormal frequency portion other than the pulse may be present in each period.

Furthermore, the signal processing unit 341 may extract a pulse wave from the oscillometric signal. The signal processing unit 341 may estimate the pulse wave from the oscillometric signal by using a predefined transfer function in response to cuff pressure corresponding to the oscillometric signal. Specifically, the signal processing unit 341 may process the oscillometric signal by dividing the oscillometric signal into pulses and applying a Fourier transform to the pulses. Specifically, the signal processing unit 341 may individually divide the oscillometric signal into pulses as illustrated in FIG. 9(a), and may apply the Fourier transform to each of the pulses as illustrated in FIG. 9(b). Accordingly, an oscillometric signal (y_{MCP}(t)) in a time domain may be transformed into an oscillometric signal (Y_{OW}(f)) in a frequency domain. As illustrated in FIG. 9(b), the signal processing unit 341 may estimate a pulse wave, for example, a waveform of central arterial pressure from the oscillometric signal by using a predefined transfer function in response to cuff pressure. In this case, the transfer function may be different depending on different cuff pressure. That is, the signal processing unit 341 may estimate central arterial pressure (\hat{X}_{AP}(f)) in the frequency domain from the oscillometric signal (Y_{OW}(f)) in the frequency domain by using an inverse transfer function (Ĝ_{MCP}⁻¹(f)) relation, and may obtain the waveform of the central arterial pressure in the time domain from the central arterial pressure (\hat{X}_{AP}(f)) in the frequency domain by applying an inverse Fourier transform. The central arterial pressure estimated as described above may be similar to actually measured central arterial pressure as illustrated in FIG. 9(b).

The analysis unit 343 may extract at least one cardiovascular health index by analyzing the characteristic factors of the oscillometric signal. The analysis unit 343 may estimate an internal diameter of a blood vessel as a cardiovascular health index in accordance with the amplitude of the oscillometric signal. The analysis unit 343 may estimate blood pressure, for example, the highest blood pressure and the lowest blood pressure as cardiovascular health indices from the type of oscillometric signal. The analysis unit 343 may estimate the cardiovascular health index based on at least one of whether an abnormal frequency portion is present within the period of each pulse or whether a change in periodicity is present in the period, from the frequency component of the oscillometric signal. For example, the analysis unit 343 may detect abnormal shaking in the frequency component, and may estimate a cardiovascular health index related to an irregular pulse from the detected abnormal shaking.

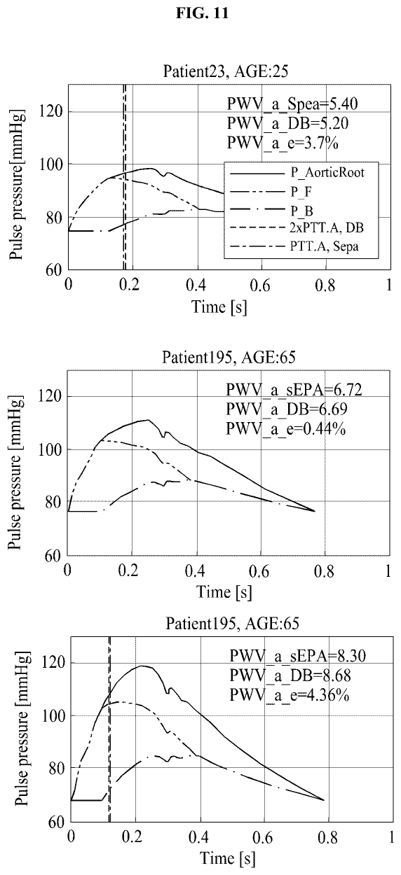

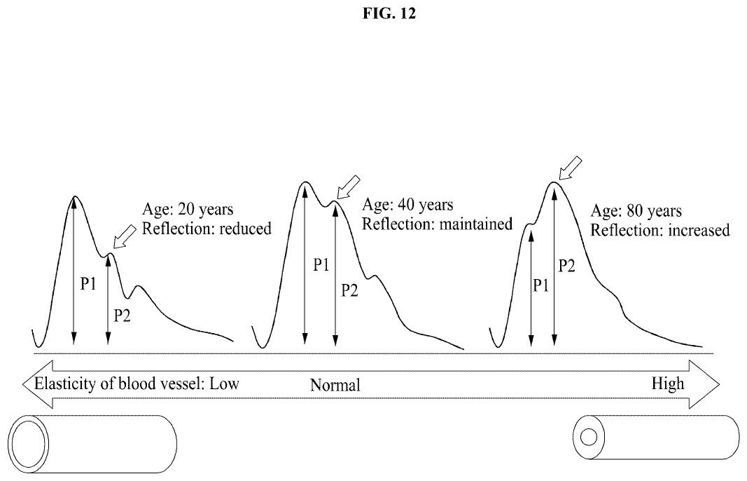

Furthermore, the analysis unit 343 may extract at least one cardiovascular health index by analyzing a pulse wave from the oscillometric signal. As illustrated in FIG. 10, the analysis unit 343 may calculate at least one of an augmentation index (AIx) (AIx=F−P/F), a stiffness index (SI) (SI=h/Δt), a time ratio (ΔT/T ratio) (ΔT/T ratio=ΔT/T), or a reflection index (RI) (RI=P/F) by using a peak point and inflection point of the pulse wave, and may estimate the elasticity of the blood vessel as a cardiovascular health index. As illustrated in FIG. 11, the analysis unit 343 may separate an advancing wave (P_F) and a reflection wave (P_B) from the pulse wave (P_AorticRoot), may calculate a pulse wave velocity (PWV) by using the reflection wave, and may estimate the elasticity of the blood vessel as a cardiovascular health index from the pulse wave velocity. In this case, the pulse wave velocity may be related to various cardiovascular health indices in addition to the elasticity of the blood vessel as in Equation 1. Accordingly, as illustrated in FIG. 12, the analysis unit 343 may estimate that the elasticity of the blood vessel is low when the reflection wave is reduced by exceeding a predetermined range, the elasticity of the blood vessel is normal when the reflection wave is maintained within the predetermined range, and the elasticity of the blood vessel is high when the reflection wave is increased by exceeding the predetermined range. Furthermore, the analysis unit 343 may estimate a blood vessel aging index corresponding the elasticity of the blood vessel.

$$PWV = \sqrt{\frac{Eh}{2 {ρ}_{fluid} R ( 1 - {v}^{2} )}} \qquad ( 1 )$$

In Equation 1, E may indicate a blood vessel elastic modulus, ρ_{fluid} may indicate blood viscosity, h may indicate the thickness of a blood vessel, R may indicate the diameter of the blood vessel, and v may indicate a Poisson's ratio.

The determination unit 345 may determine a cardiovascular disease by analyzing the tendency of cardiovascular health indices that are accumulated for a predetermined interval. In general, the cardiovascular disease corresponds to an anaplastia disease. Accordingly, the determination unit 345 may determine a cardiovascular disease by analyzing the tendency of cardiovascular health indices that are accumulated for a predetermined interval over a flow of time. Accordingly, the tendency of the cardiovascular health indices may be used for the prevention and early diagnosis of a cardiovascular disease.

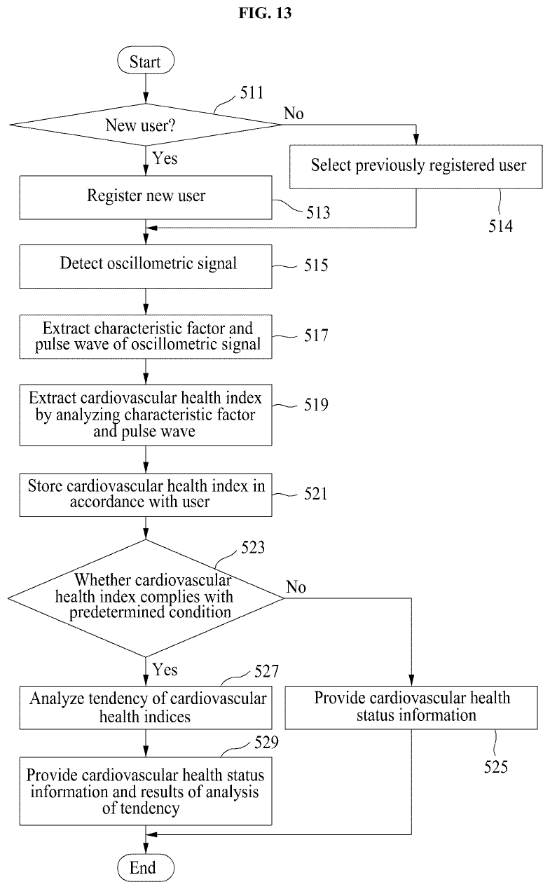

FIG. 13 is a diagram schematically illustrating an operating method of the electronic device 100 according to various embodiments.

Referring to FIG. 13, in step 511, the electronic device 100 may determine whether a user who approaches the electronic device 100 is a new user. In this case, the processor 140 may confirm identification information of the user, and may determine whether the user is a user who has been previously registered based on the identification information. When it is determined that the user is a new user in step 511, the electronic device 100 may register the new user therewith in step 513. In this case, the processor 140 may register the new user while storing the identification information of the new user. When it is determined that the user is not a new user in step 511, the electronic device 100 may select a previously registered user in step 514. In this case, the processor 140 may select the identification information of the previously registered user.

Next, in step 515, the electronic device 100 may detect an oscillometric signal in accordance with the user. In this case, the processor 140 may detect the oscillometric signal from pressure vibration that is generated from a cuff in response to cuff pressure by using the oscillometric blood pressure gauge 10 as illustrated in FIG. 4. Furthermore, the processor 140 may obtain time information indicating a date and time of the measurement of the oscillometric signal along with the oscillometric signal.

Next, in step 517, the electronic device 100 may extract at least one characteristic factor and pulse wave of the oscillometric signal. In this case, as illustrated in at least one of FIG. 5, 7, or 8, the processor 140 may extract at least one characteristic factor of the oscillometric signal. The characteristic factor may include at least one of the amplitude, type, or frequency component of the oscillometric signal. Furthermore, the processor 140 may extract a pulse wave from the oscillometric signal. The processor 140 may estimate the pulse wave from the oscillometric signal by using a predefined transfer function in response to the cuff pressure corresponding to the oscillometric signal. Specifically, the processor 140 may process the oscillometric signal by dividing the oscillometric signal into pulses and applying a Fourier transform to each of the pulses. Specifically, the processor 140 may individually divide the oscillometric signals into the pulses as illustrated in FIG. 9(a), and may apply the Fourier transform to each of the pulses as illustrated in FIG. 9(b). Accordingly, the oscillometric signal (y_{MCP}(t)) in the time domain may be transformed into the oscillometric signal (Y_{OW}(f)) in the frequency domain. The processor 140 may estimate the pulse wave from the oscillometric signal by using the predefined transfer function in response to the cuff pressure, as illustrated in FIG. 9(b). In this case, the transfer function may be different depending on different cuff pressure.

Next, in step 519, the electronic device 100 may extract a cardiovascular health index by analyzing the characteristic factor and the pulse wave. In this case, the processor 140 may extract at least one cardiovascular health index by analyzing the characteristic factor of the oscillometric signal. The processor 140 may estimate an internal diameter of a blood vessel as a cardiovascular health index in accordance with amplitude of the oscillometric signal. The processor 140 may estimate blood pressure, for example, the highest blood pressure and the lowest blood pressure as cardiovascular health indices from the type of oscillometric signal. The processor 140 may estimate the cardiovascular health index based on at least one of whether an abnormal frequency portion is present within the period of each pulse or whether a change in periodicity is present in the period from a frequency component of the oscillometric signal. For example, the processor 140 may detect abnormality shaking in the frequency component, and may estimate a cardiovascular health index related to an irregular pulse from the detected abnormality shaking. Furthermore, the processor 140 may extract at least one cardiovascular health index by analyzing a pulse wave from the oscillometric signal. As illustrated in FIG. 10, the processor 140 may calculate at least one of an augmentation index (AIx=F−P/F), a stiffness index (SI=h/Δt), a time ratio (ΔT/T ratio=ΔT/T), or a reflection index (RI=P/F) by using a peak point and inflection point of the pulse wave, and may estimate the elasticity of a blood vessel as a cardiovascular health index based on the extracted at least one. As illustrated in FIG. 11, the processor 140 may separate an advancing wave (P_F) and a reflection wave (P_B) from the pulse wave (P_AorticRoot), may calculate a pulse wave velocity by using the reflection wave, and may estimate the elasticity of the blood vessel as a cardiovascular health index from the pulse wave velocity.

Next, in step 521, the electronic device 100 may store the cardiovascular health index in accordance with the user. In this case, the processor 140 may store the cardiovascular health index in the memory 130 along with time information in accordance with the identification information of the user. When the user is a previously registered user, the processor 140 may accumulate and store the cardiovascular health indices and the time information in accordance with the identification information of the user.

Next, in step 523, the electronic device 100 may determine whether the stored cardiovascular health index complies with a predetermined condition. In this case, the processor 140 may determine whether the cardiovascular health indices of the user have been accumulated for a predetermined interval.

If it is determined that the cardiovascular health indices have not been accumulated for the predetermined interval in step 523, in step 525, the electronic device 100 may provide cardiovascular health status information. In this case, the processor 140 may determine the cardiovascular health status information based on the cardiovascular health index extracted in step 519, and may provide the cardiovascular health status information through the output module 120. That is, the processor 140 may provide basic information, such as blood pressure and a pulse.

If it is determined that the cardiovascular health indices have been accumulated for the predetermined interval in step 523, in step 527, the electronic device 100 may analyze the tendency of the accumulated cardiovascular health indices. In this case, the processor 140 may analyze the tendency based on a change in the accumulated cardiovascular health indices according to a flow of time for the predetermined interval. Thereafter, in step 529, the electronic device 100 may provide the results of the analysis of the cardiovascular health status information and tendency. In this case, the processor 140 may determine the cardiovascular health status information based on the cardiovascular health index extracted in step 519, and may provide the cardiovascular health status information through the output module 120. Moreover, the processor 140 may provide whether a cardiovascular disease is present based on the results of the analysis of the tendency. That is, the processor 140 may provide deep information along with the basic information.

According to the present disclosure, the electronic device 100 may extract a cardiovascular health status by using an oscillometric signal. That is, the electronic device 100 may check the cardiovascular health status and the course of a change thereof by analyzing the oscillometric signal by using an automatic blood pressure gauge that is commonly used, that is, the oscillometric blood pressure gauge 10. Accordingly, the electronic device 100 may be applied to all fields in which the automatic blood pressure gauge is used. For example, when blood pressure of a patient who periodically visits a hospital is measured by using the automatic blood pressure gauge, the electronic device 100 can determine a cardiovascular disease by extracting a cardiovascular health status in addition to blood pressure and checking the course of a change thereof. As another example, when the automatic blood pressure gauge is periodically used in a home, the electronic device 100 can determine a cardiovascular disease by extracting a cardiovascular health status in addition to blood pressure and checking the course of a change thereof. Accordingly, a cardiovascular disease can be prevented and early diagnosed. Moreover, even people whose blood pressure is measured high due to a white coat syndrome when the blood pressure is measured in a hospital can check their cardiovascular health statuses in a comfortable environment, such as a home. Furthermore, patients who belong to a high risk group for a cardiovascular disease can previously recognize the dangerousness of the cardiovascular disease through consistent measurement even in homes. Such an electronic device 100 may be applied to all types of devices which detect an oscillometric signal, such as an automatic blood pressure gauge.

To sum up, the present disclosure provides a device and method for extracting a cardiovascular health status by using an oscillometric signal.

According to an embodiment of the present disclosure, a method of extracting, by the electronic device 100, a cardiovascular health status by using an oscillometric signal may include steps of extracting at least one characteristic factor of an oscillometric signal, extracting a pulse wave from the oscillometric signal, and extracting at least one cardiovascular health index by analyzing the characteristic factor and the pulse wave.

According to various embodiments, the characteristic factor may include amplitude of the oscillometric signal. The step of extracting the cardiovascular health index may include a step of estimating, as the cardiovascular health index, an internal diameter of a blood vessel corresponding to the amplitude.

According to various embodiments, the characteristic factor may include the type of oscillometric signal. The step of extracting the cardiovascular health index may include a step of estimating blood pressure as the cardiovascular health index from the type.

According to various embodiments, the characteristic factor may include a frequency component of the oscillometric signal. The step of extracting the cardiovascular health index may include a step of estimating the cardiovascular health index based on at least one of whether an abnormal frequency portion is present in a period of each pulse or whether a change in periodicity is present in the period from the frequency component.

According to various embodiments, the step of extracting the pulse wave may include a step of estimating the pulse wave from the oscillometric signal by using a predefined transfer function based on cuff pressure corresponding to the oscillometric signal.

According to various embodiments, the step of extracting the cardiovascular health index may include steps of calculating an augmentation index (Aix) based on a peak point and inflection point of the pulse wave, and estimating elasticity of a blood vessel as the cardiovascular health index from the augmentation index.

According to various embodiments, the step of extracting the cardiovascular health index may include steps of separating an advancing wave and a reflection wave from the pulse wave, calculating a pulse wave velocity (PWV) by using the reflection wave, and estimating elasticity of a blood vessel as the cardiovascular health index from the pulse wave velocity.

According to various embodiments, the method of extracting the cardiovascular health status may further include a step of determining a cardiovascular disease by analyzing a tendency of accumulated cardiovascular health indices of a user when the cardiovascular health indices are accumulated for a predetermined interval.

According to an embodiment of the present disclosure, the electronic device 100 for extracting a cardiovascular health status by using an oscillometric signal may include memory 130, and a processor 140 connected to the memory 130 and configured to execute at least one instruction stored in the memory 130. The processor 140 may be configured to extract at least one characteristic factor of an oscillometric signal, extract a pulse wave from the oscillometric signal, and extract at least one cardiovascular health index by analyzing the characteristic factor and the pulse wave.

According to various embodiments, the characteristic factor may include amplitude of the oscillometric signal. The processor 140 may be configured to estimate, as the cardiovascular health index, an internal diameter of a blood vessel corresponding to the amplitude.

According to various embodiments, the characteristic factor may include the type of oscillometric signal. The processor 140 may be configured to estimate blood pressure as the cardiovascular health index from the type.

According to various embodiments, the characteristic factor may include a frequency component of the oscillometric signal. The processor 140 may be configured to estimate the cardiovascular health index based on at least one of whether an abnormal frequency portion is present in a period of each pulse or whether a change in periodicity is present in the period from the frequency component.

According to various embodiments, the processor 140 may be configured to estimate the pulse wave from the oscillometric signal by using a predefined transfer function based on cuff pressure corresponding to the oscillometric signal.

According to various embodiments, the processor 140 may be configured to calculate an augmentation index (Aix) based on a peak point and inflection point of the pulse wave, and estimate elasticity of a blood vessel as the cardiovascular health index from the augmentation index.

According to various embodiments, the processor 140 may be configured to separate an advancing wave and a reflection wave from the pulse wave, calculate a pulse wave velocity (PWV) by using the reflection wave, and estimate elasticity of a blood vessel as the cardiovascular health index from the pulse wave velocity.

According to various embodiments, the processor 140 may be configured to determine a cardiovascular disease by analyzing a tendency of accumulated cardiovascular health indices of a user when the cardiovascular health indices are accumulated for a predetermined interval.

The aforementioned system may be implemented as a hardware component, a software component and/or a combination of a hardware component and a software component. For example, the system and component described in the embodiments may be implemented by using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing or responding to an instruction. The processing device may perform an operating system (OS) and one or more software applications executed on the OS. Furthermore, the processing device may access, store, manipulate, process and generate data in response to the execution of software. For convenience of understanding, one processing device has been illustrated as being used, but a person having ordinary knowledge in the art may understand that the processing device may include a plurality of processing elements and/or a plurality of types of processing elements. For example, the processing device may include a plurality of processors or one processor and one controller. Furthermore, another processing configuration, such as a parallel processor, is also possible.

Software may include a computer program, a code, an instruction or a combination of one or more of them and may configure a processing device so that the processing device operates as desired or may instruct the processing devices independently or collectively. The software and/or the data may be embodied in any type of machine, a component, a physical device, or a computer storage medium or device in order to be interpreted by the processing device or to provide an instruction or data to the processing device. The software may be distributed to computer systems connected over a network and may be stored or executed in a distributed manner. The software and the data may be stored in one or more computer-readable recording media.

The method according to the embodiments may be implemented in the form of a program instruction executable by various computer means, and may be stored in a computer-readable medium. In this case, the medium may continue to store a program executable by a computer or may temporarily store the program for execution or download. Furthermore, the medium may be various recording means or storage means having a form in which one or a plurality of pieces of hardware has been combined. The medium is not limited to a medium that is directly connected to a computer system, but may be ones that are distributed and present in a network. Examples of the medium may be magnetic media such as a hard disk, a floppy disk, and a magnetic tape, optical media such as CD-ROM and a DVD, magneto-optical media such as a floptical disk, and ones configured to store a program command, including ROM, RAM, and a flash memory. Furthermore, examples of another medium may include an app store in which apps are distributed, a site in which other various pieces of software are supplied or distributed, and recording media and/or storage media that are managed in a server.

Various embodiments of this document and the terms used in the embodiments are not intended to limit the technology described in this document to a specific embodiment, but should be construed as including various changes, equivalents and/or alternatives of a corresponding embodiment. In relation to the description of the drawings, similar reference numerals may be used in similar components. An expression of the singular number may include an expression of the plural number unless clearly defined otherwise in the context. In this document, an expression, such as “A or B”, “at least one of A and/or B”, “A, B, or C” or “at least one of A, B and/or C”, may include all of possible combinations of items listed together. Expressions, such as “a first,” “a second,” “the first”, and “the second”, may modify corresponding components regardless of its sequence or importance, and are used to only distinguish one component from another component and do not limit corresponding components. When it is described that one (e.g., a first) component is “(functionally or communicatively) connected to” or “coupled with” the other (e.g., a second) component, one component may be directly connected to another component or may be connected to another component through another component (e.g., a third component).

The term “module” used in the present disclosure includes a unit configured as hardware, software or firmware, and may be interchangeably used with a term, such as logic, a logical block, a part or a circuit. The module may be an integrated part, a minimum unit to perform one or more functions, or a part thereof. For example, the module may be configured as an application-specific integrated circuit (ASIC).

According to various embodiments, each (e.g., a module or a program) of the aforementioned elements may include a single entity or a plurality of entities. According to various embodiments, one or more of the aforementioned components or steps may be omitted or one or more other components or steps may be added. Alternatively or additionally, a plurality of components (e.g., modules or programs) may be integrated into a single component. In such a case, the integrated component may identically or similarly perform a function performed by a corresponding one of the plurality of components before one or more functions of each of the plurality of components are integrated. According to various embodiments, steps performed by a module, a program or another component may be executed sequentially, in parallel, iteratively or heuristically, or one or more of the steps may be executed in different order or may be omitted, or one or more other steps may be added.

1. A method of extracting, by an electronic device, a cardiovascular health status by using an oscillometric signal, the method comprising:
extracting at least one characteristic factor of an oscillometric signal;
extracting a pulse wave from the oscillometric signal; and
extracting at least one cardiovascular health index by analyzing the characteristic factor and the pulse wave.

2. The method of claim 1, wherein the characteristic factor comprises amplitude of the oscillometric signal, and
wherein the extracting of the cardiovascular health index comprises estimating, as the cardiovascular health index, an internal diameter of a blood vessel corresponding to the amplitude.

3. The method of claim 1, wherein the characteristic factor comprises a type of the oscillometric signal, and
wherein the extracting of the cardiovascular health index comprises estimating blood pressure as the cardiovascular health index from the type.

4. The method of claim 1, wherein the characteristic factor comprises a frequency component of the oscillometric signal, and
wherein the extracting of the cardiovascular health index comprises estimating the cardiovascular health index based on at least one of whether an abnormal frequency portion is present in a period of each pulse or whether a change in periodicity is present in the period from the frequency component.

5. The method of claim 1, wherein the extracting of the pulse wave comprises estimating the pulse wave from the oscillometric signal by using a predefined transfer function based on cuff pressure corresponding to the oscillometric signal.

6. The method of claim 1, wherein the extracting of the cardiovascular health index comprises:
calculating an augmentation index (Aix) based on a peak point and inflection point of the pulse wave; and
estimating elasticity of a blood vessel as the cardiovascular health index from the augmentation index.

7. The method of claim 1, wherein the extracting of the cardiovascular health index comprises:
separating an advancing wave and a reflection wave from the pulse wave;
calculating a pulse wave velocity (PWV) by using the reflection wave; and
estimating elasticity of a blood vessel as the cardiovascular health index from the pulse wave velocity.

8. The method of claim 1, further comprising:
determining a cardiovascular disease by analyzing a tendency of accumulated cardiovascular health indices of a user when the cardiovascular health indices are accumulated for a predetermined interval.

9. An electronic device for extracting a cardiovascular health status by using an oscillometric signal, the electronic device comprising:
memory; and
a processor connected to the memory and configured to execute at least one instruction stored in the memory,
wherein the processor is configured to:
extract at least one characteristic factor of an oscillometric signal,
extract a pulse wave from the oscillometric signal, and
extract at least one cardiovascular health index by analyzing the characteristic factor and the pulse wave.

10. The electronic device of claim 9, wherein the characteristic factor comprises amplitude of the oscillometric signal, and
wherein the processor is configured to estimate, as the cardiovascular health index, an internal diameter of a blood vessel corresponding to the amplitude.

11. The electronic device of claim 9, wherein the characteristic factor comprises a type of the oscillometric signal, and
wherein the processor is configured to estimate blood pressure as the cardiovascular health index from the type.

12. The electronic device of claim 9, wherein the characteristic factor comprises a frequency component of the oscillometric signal, and
wherein the processor is configured to estimate the cardiovascular health index based on at least one of whether an abnormal frequency portion is present in a period of each pulse or whether a change in periodicity is present in the period from the frequency component.

13. The electronic device of claim 9, wherein the processor is configured to estimate the pulse wave from the oscillometric signal by using a predefined transfer function based on cuff pressure corresponding to the oscillometric signal.

14. The electronic device of claim 9, wherein the processor is configured to:
calculate an augmentation index (Aix) based on a peak point and inflection point of the pulse wave, and
estimate elasticity of a blood vessel as the cardiovascular health index from the augmentation index.

15. The electronic device of claim 9, wherein the processor is configured to:
separate an advancing wave and a reflection wave from the pulse wave,
calculate a pulse wave velocity (PWV) by using the reflection wave, and
estimate elasticity of a blood vessel as the cardiovascular health index from the pulse wave velocity.

16. The electronic device of claim 9, wherein the processor is configured to determine a cardiovascular disease by analyzing a tendency of accumulated cardiovascular health indices of a user when the cardiovascular health indices are accumulated for a predetermined interval.

17. A non-transitory computer-readable recording medium in which a computer program for executing a method of extracting a cardiovascular health status by using an oscillometric signal in an electronic device has been stored, the method comprising:
extracting at least one characteristic factor of an oscillometric signal;
extracting a pulse wave from the oscillometric signal; and
extracting at least one cardiovascular health index by analyzing the characteristic factor and the pulse wave.

18. The non-transitory computer-readable recording medium of claim 17, wherein the characteristic factor comprises at least one of amplitude, a type, or a frequency component of the oscillometric signal.

19. The non-transitory computer-readable recording medium of claim 17, wherein the extracting of the cardiovascular health index comprises estimating elasticity of a blood vessel as the cardiovascular health index, based on at least one of an augmentation index that is calculated by using a peak point and inflection point of the pulse wave, or a pulse wave velocity that is calculated by using an advancing wave and a reflection wave separated from the pulse wave.

20. The non-transitory computer-readable recording medium of claim 17, wherein the method further comprises determining a cardiovascular disease by analyzing a tendency of accumulated cardiovascular health indices of a user when the cardiovascular health indices are accumulated for a predetermined interval.
